SEQUENCE LISTING (12 sequences)

SEQ ID NO: 1 - DNA, 867 bp, Homo sapiens
atgcgggaccggctgccagacctgacggcgtgtaggaagaatgatgatggagacacagttgttgtggttgagaaagatcatttcatggatgatttcttccatcaggtggaggagattagaaacagtattgataaaataactcaatatgttgaagaagtaaagaaaaaccacagcatcattctttctgcaccaaacccggaaggaaaaataaaagaagagcttgaagatctgaacaaagaaatcaagaaaactgcgaataaaattcgagccaagttaaaggctattgaacaaagttttgatcaggatgagagtgggaaccggacttcagtggatcttcggatacgaagaacccagcattcggtgctgtctcggaagtttgtggaagccatggcggagtacaatgaggcacagactctgtttcgggagcggagcaaaggccgcatccagcgccagctggagataactgggagaaccaccacagacgacgagctagaagagatgctggagagcgggaagccatccatcttcacttccgacattatatcagattcacaaattactagacaagctctcaatgaaatcgagtcacgtcacaaggacatcatgaagctggagaccagcatccgagagttgcatgagatgttcatggacatggctatgtttgtggagactcagggtgaaatgatcaacaacatagaaagaaatgttatgaatgccacagactatgtagaacacgctaaagaagaaacaaaaaaagctatcaaatatcagagcaaggcaagaaggaaaaagtggataattattgctgtgtcagtggttctggttgtcataatcgttctaattattggcttgtcagttggcaaatga

SEQ ID NO: 2 - DNA, 834 bp, Homo sapiens
atgcgggaccggctgccagacctgacggcgtgtaggaagaatgatgatggagacacagttgttgtggttgagaaagatcatttcatggatgatttcttccatcaggtggaggagattagaaacagtattgataaaataactcaatatgttgaagaagtaaagaaaaaccacagcatcattctttctgcaccaaacccggaaggaaaaataaaagaagagcttgaagatctgaacaaagaaatcaagaaaactgcgaataaaattcgagccaagttaaaggctattgaacaaagttttgatcaggatgagagtgggaaccggacttcagtggatcttcggatacgaagaacccagcattcggtgctgtctcggaagtttgtggaagccatggcggagtacaatgaggcacagactctgtttcgggagcggagcaaaggccgcatccagcgccagctggagataactgggagaaccaccacagacgacgagctagaagagatgctggagagcgggaagccatccatcttcacttccgacattatatcagattcacaaattactagacaagctctcaatgaaatcgagtcacgtcacaaggacatcatgaagctggagaccagcatccgagagttgcatgagatgttcatggacatggctatgtttgtggagactcagggtgaaatgatcaacaacatagaaagaaatgttatgaatgccacagactatgtagaacacgctaaagaagaaacaaaaaaagctatcaaatatcagagcaaggcaagaaggcaacaacattgtcatagcaaccatatcccaagagccatttatccttga

SEQ ID NO: 3 - DNA, 702 bp, Homo sapiens
aggaagccgactaggcgaattcacttactgaccggcctgggctgctctgagacatggaggaagccagtgaaggtggaggaaatgatcgtgtgcggaacctgcaaagtgaggtggagggagttaagaatattatgacccagaatgtggagcggatcctggcccggggggaaaacttggaacatctccgcaacaagacagaggatctggaagccacatctgagcacttcaagacgacatcgcagaaggtggctcggaaattctggtggaagaacgtgaagatgattgtccttatctgcgtgattgtttttatcatcatcctcttcattgtgctctttgccactggtgccttctcttaagtaacagggaacctctcccacctgcccttcttttcagggacaaccctccataaatgtgtgccaagagggtctcctttcctgtcttcctctacagagaatgctgctcggtcctcctacccctcttcccgaggccctgctgccacgttgtatgccccagaaggtaccttggtcccccggaaggagagaaaaaagagagatggactgtggctgcatttcttgggtccttagagtgggctggagagacctagagggcccagcatgtggctgggaaactgttggtggccagtgggtaataaagacctttcagtatccctaaaaaaaaaaaaaaaaaaaaaaaaaaaaaaaa

SEQ ID NO: 4 - DNA, 303 bp, Rattus norvegicus
atggaggccagtgggagtgccggaaatgaccgagtcaggaacctgcagagtgaggtggagggagtcaagaatattatgacccagaatgtggagcggatcttggcccgaggggagaacctggaccatctccgaaacaagacagaggacttggaagccacgtctgaacacttcaagacgacgtcccagaaggtggcccggaagttctggtggaagaatgtgaagatgattgtcatcatctgtgtgattgtccttatcattctcatcctcatcatactctttgctacgggcaccatccccacttaa

SEQ ID NO: 5 - DNA, 3003 bp, Rattus norvegicus
atcgccaccgcagcctaggcagagccagtcggcccaggcccctgtctctgcctggcctcagctccccgccggcccgccgcgcaccttacccgcacatcccttggaggtctagccgggtgcccccagaccccggcctccagcacagaggcaagaggcagagagcagcagcgagggcgaggacgaagaaggggaggaggagctcgtcgcaggatgaaggatcggactcaggagctgcggagtgcaaaagacagtgacgatgaagaggaagtggttcatgtggatcgagaccactttatggatgagttctttgagcaggtggaagagatccgaggctgcatcgagaaactgtccgaggatgtggagcaagtgaagaaacagcacagtgccattcttgctgcccccaaccccgatgagaagactaaacaggagctggaggacctcacggcagacatcaaaaagacggcaaacaaggtccggtccaagttgaaagcgatcgagcagagcattgagcaggaagaggggttgaatcgttcttctgcagacctgcgtatccgtaagacccagcactccacactctcacggaagttcgtggaggtaatgaccgaatataatgcaactcagtctaagtaccgggaccgctgcaaggaccgtatccagaggcagctggagatcactggcaggactactaccaacgaagagctggaagacatgttggaaagcgggaagctggccatcttcacggacgacatcaaaatggactcgcagatgacaaagcaagccctgaatgagatagagacaaggcacaatgagatcatcaaactggaaaccagcatccgagagctgcacgacatgtttgtggacatggccatgctcgtggagagccagggtgagatgatcgaccgaattgagtacaatgtggaacattctgtggactacgtggagcgagccgtgtccgacaccaagaaagctgtgaaatatcagagcaaggccaggaggaagaaaattatgatcatcatttgctgtgtggtgctgggggtggtcttggcgtcatctattggggggacactgggcttgtaggcccctacccttctcttccccaggaccctccccacacatcgggagcaatacccccaccaccctttcactctttcccctgctccaagctcactcccaaaacagacccaggcagttccagcctctctcaccctcacgcagaccctggagtccctggctctcaccttgccatggatccccctccaccttgccgcacatagatagcagcaggcgtgatcacacatgcacaccaacatgcatgccgagggcacatgctcaagacgtgtggacaccccagcgtgtgtgtacttgtgtagatgtatgtagatgccctgaacctcttcttgctgccaccttcatcctgtgtggtctgaacttccctctctagccggttctgtgctgactgtagcagcctaccatggcccaacctgttctgtgtgaatagacatggtgtgtatgtgtgtgtgtgtgtgtgtgtgtgtgtgtgtgtgtgtgtgtgtgtgtgtagatctgtgcccatacaatgtatgtacatggagttttgtgaatgtgaaaacccaaagcatttcctccaccttctctcctatctgttacatttggagtgggaggcatatgtgaggataatttacattctcagaggatccagggtggcccggtgaccatgccacacatcttttgtgatgtagaacaaactttcctggcttggttactgccaggggtcacatcatgtcctgaggattctctctctcagcctctctctctgtctctctgtccctcagtctctgtctctgtctctgtctctgtgtgtgtgtgtgtgtgtgtgtgtgtgtgtgtgtccacatgtatgtaacccctagattagctgtgctgaggaaggaggctgcctgtccattccagaatattctgctgcagggcccagcacctcctgcccccaccttggcttctttggagatgtcagaggaggccccaggatctagttcccttcttctttcagcccagccagaactcccattaactcagatgtttctagcagcctccctgacaccccagtcatggtgttcacaaggacagggctgtggctggcatgctgctcctgcctctttgaatggcactgggcccacagctgcccaccactgagcctcaaacagatgagccgtccctgagagtctgctgtaacccagctttctaagacccacacctcacacgatggacatgttcgttgactgctgtccacatgtgtatcctggatggttgtctgggggctggtgtctgttgcatgttctcagatgtgtgctgcgtgccctccacacaccccaaaccatcaagcccaattatactcccttggtggtcccaggcccaagccaggactcaatgcttccattctccctttccttgcctcttacaaagcacctgcgtgtccatctcatgtgcccatgggtcatcatgctcccgtcataccttgagcgtgtacacatgtgtgttctatgcactcgccctgccctacctacccaacagaggacaagatggtcggcccctagctcccttctcccccacctagcctttccccacgccctgctgtgcagctgtgtgcgttggtgtgtttctgtgtcgctggcgtgtcatgtgatgtagccatgtttgctgacatgagcccctgcccccttctctttttctccattggtttctagaactctcttcctcccctcccctgagggacaggactcctggggcctagctgggggcccgagcctggccaccctcctgttagccctcagagtcttatttctctctattggtgaccaagttgcaaatggataaaatacaggaaattctgaccccctgccccagacctgcatgtcctgtccccagtgcccccgaaccccatcctgggccgggttgggcctggtgggacgggagaaatagcaactaatccaacagcgaaaaaaaaaa

SEQ ID NO: 6 - DNA, 825 bp, Rattus norvegicus
atgcgggaccggctgccggacctcacggcgtgtaggaaaagcgacgatggagacaatgctgtcatcatcacggtggagaaggaccacttcatggatgccttcttccatcaggtggaggagattcgaagcagcatagccaggattgctcagcacgtggaggatgtgaagaagaaccacagcatcatcctctctgccccaaacccagaaggaaaaataaaagaagagctggaggacctgaacaaagagatcaagaaaactgctaacaggatccggggcaagctgaaggctattgagcagagttgtgatcaggacgagaatgggaaccgaacttcagtggatctgcggatacgaaggacccagcactcagtgctgtcacggaagtttgtggacgtcatgacagaatacaatgaagcacagatcctgtttcgggagcgaagcaaaggccgaatccagcgccagctagagatcactgggaggaccaccactgacgaggagctggaggagatgctggagagcgggaagccgtccatcttcatctcggacattatatcagattcacagattactaggcaagctctcaatgagatcgagtcacgccacaaagacatcatgaagctggagaccagcatccgagagctgcacgagatgttcatggatatggccatgtttgtcgagactcagggtgaaatggtcaacaacattgagagaaacgtggtgaactccgtagattacgtggagcacgccaaggaagagactaagaaagccatcaaataccagagcaaggccagacggggtgtgctctgtgctctcggccgacagtgctga

SEQ ID NO: 7 - DNA, 870 bp, Rattus norvegicus
atgcgggaccggctgccggacctcacggcgtgtaggaaaagcgacgatggagacaatgctgtcatcatcacggtggagaaggaccacttcatggatgccttcttccatcaggtggaggagattcgaagcagcatagccaggattgctcagcacgtggaggatgtgaagaagaaccacagcatcatcctctctgccccaaacccagaaggaaaaataaaagaagagctggaggacctgaacaaagagatcaagaaaactgctaacaggatccggggcaagctgaaggctattgagcagagttgtgatcaggacgagaatgggaaccgaacttcagtggatctgcggatacgaaggacccagcactcagtgctgtcacggaagtttgtggacgtcatgacagaatacaatgaagcacagatcctgtttcgggagcgaagcaaaggccgaatccagcgccagctagagatcactgggaggaccaccactgacgaggagctggaggagatgctggagagcgggaagccgtccatcttcatctcggacattatatcagattcacagattactaggcaagctctcaatgagatcgagtcacgccacaaagacatcatgaagctggagaccagcatccgagagctgcacgagatgttcatggatatggccatgtttgtcgagactcagggtgaaatggtcaacaacattgagagaaacgtggtgaactccgtagattacgtggagcacgccaaggaagagactaagaaagccatcaaataccagagcaaggccagacggaaggtgatgttcgtcctcatctgtgtagtcactttgcttgtgatccttggaattattctagcaacagcattgtcatag

SEQ ID NO: 8 - DNA, 1109 bp, Homo sapiens
gcggggcctgaggcggagaccggagagcccgcggcccggccggaggcagctcgggacaggcttgagcggcggggcgcgctgcccggccggcggggatgcgggaccggctgccagacctgacggcgtgtaggaagaatgatgatggagacacagttgttgtggttgagaaagatcatttcatggatgatttcttccatcaggtggaggagattagaaacagtattgataaaataactcaatatgttgaagaagtaaagaaaaaccacagcatcattctttctgcaccaaacccggaaggaaaaataaaagaagagcttgaagatctgaacaaagaaatcaagaaaactgcgaataaaattgcagccaagttaaaggctattgaacaaagttttgatcaggatgagagtgggaaccggacttcagtggatcttcggatacgaagaacccagcattcggtgctgtctcggaagtttgtggaagccatggcggagtacaatgaggcacagactctgtttcgggagcggagcaaaggccgcatccagcgccagctggagataactgggagaaccaccacagacgacgagctagaagagatgctggagagcgggaagccatccatcttcacttccgacattatatcagattcacaaattactagacaagctctcaatgaaatcgagtcacgtcacaaggacatcatgaagctggagaccagcatccgagagttgcatgagatgttcatggacatggctatgtttgtggagactcagggtgaaatgatcaacaacatagaaagaaatgttatgaatgccacagactatgtagaacacgctaaagaagaaacaaaaaaagctatcaaatatcagagcaaggcaagaaggaaaaagtggataattattgctgtgtcagtggttctggttgtctatcgtctatttggcttgtcgttggaatatgttgtacgcagtgctgcctctctgccagggtggggaaattgatgttcattatattgaagtttgtttattgattctcacacatcaaaccaccaagattcctgctgcaatgaaccaaatcagcatcctgtcatttcgtgaatgaatctcagacgctgt

SEQ ID NO: 9 - protein, 288 aa, Rattus norvegicus
MRDRLPDLTACRKSDDGDNAVIITVEKDHFMDAFFHQVEEIRSSIARIAQHVEDVKKNHSIILSAPNPEGKIKEELEDLNKEIKKTANIRGKLKAIEQSCDQDENGNRTSVDLRIRRTQHSVLSRKFVDVMTEYNEAQILFRERSKGRIQRQLEITGRTTDEELEEMLESGKPSIFISDIISDSQITRQALNEIESRHKDIMKLETSIRELHEMFMDMAMFVETQGEMVNNIERNVVNSVDYVEHAKEETKKAIKYQSKARRKKWIIAAVVVAVIAVLALIIGLTVGK

SEQ ID NO: 10 - protein, 287 aa, Rattus norvegicus
MRDRLPDLTACRKSDDGDNAVIITVEKDHFMDAFFHQVEEIRSSIARIAQHVEDVKKNHSIILSAPNPEGKIKEELEDLNKEIKKTANIRGKLKAIEQSCDQDENGNRTSVDLRIRRTQHSVLSRKFVDVMTEYNEAQILFRERSKGRIQRQLEITGRTTDEELEEMLESGKPSIFISDIISDSQITRQALNEIESRHKDIMKLETSIRELHEMFMDMAMFVETQGEMVNNIERNVVNSVDYVEHAKEETKKAIKYQSKARRKVMFVLICVVTLLVILGIILATALS

SEQ ID NO: 11 - protein, 272 aa, Rattus norvegicus
MRDRLPDLTACRKSDDGDNAVIITVEKDHFMDAFFHQVEEIRSSIARIAQHVEDVKKNHSIILSAPNPEGKIKEELEDLNKEIKKTANIRGKLKAIEQSCDQDENGNRTSVDLRIRRTQHSVLSRKFVDVMTEYNEAQILFRERSKGRIQRQLEITGRTTDEELEEMLESGKPSIFISDIISDSQITRQALNEIESRHKDIMKLETSIRELHEMFMDMAMFVETQGEMVNNIERNVVNSVDYVEHAKEETKKAIKYQSKARRGVLCALGRQC

SEQ ID NO: 12 - protein, 299 aa, Homo sapiens [sequence omitted]

(19) United States
(12) Patent Application Publication
(10) Pub. No.: US 2004/0091483 A1
(43) Pub. Date: May 13, 2004

(54) SNAREs and methods of controlling cytokinesis

(76) Inventors: Thomas Weimbs, Shaker Heights, OH (US); Seng Hui Low, Shaker Heights, OH (US)

Correspondence Address: Pepper Hamilton LLP, One Mellon Center, 50th Floor, 500 Grant Street, Pittsburgh, PA, 15219, US

(21) Appl. No.: 10/364,141

(22) Filed: Feb. 10, 2003

Related U.S. Application Data
(60) Provisional application No. 60/355,323, filed on Feb. 8, 2002.

(51) Int. Cl. A61K 39/395

(52) U.S. Cl. 424/146.1

(57) ABSTRACT

Two members of the SNARE membrane fusion machinery, syntaxin 2 and endobrevin/VAMP-8, have been found to be important to cytokinesis in mammalian cells. Inhibition of syntaxin 2 and endobrevin/VAMP-8 function by over-expression of non-membrane anchored mutants of these proteins causes failure of cytokinesis leading to the formation of binucleated cells. Time-lapse microscopy shows that only midbody abscission is prevented by over-expression of these non-membrane anchored mutants, and that other cellular events preceeding midbody abscission, such as furrowing, are unaffected.

### CROSS REFERENCES

This application claims priority to U.S. Provisional Application Serial No. 60/355,323, filed Feb. 8, 2002, the contents of which are incorporated herein by reference in its entirety.

### BACKGROUND TO THE INVENTION

Cytokinesis, the division of a cell into two daughter cells is a fundamental process in biology common to all organisms. In animal cells, cytokinesis is a multi-step process that involves the assembly of an actin/myosin-dependent contractile ring that guides the invagination of the plasma membrane leading to the formation of a cleavage furrow. Furrowing proceeds until the cytoplasm is constricted to a narrow bridge, termed the midbody, that contains the remnants of the spindle microtubules and connects the two prospective daughter cells. The terminal step of cytokinesis is the abscission of the midbody which leads to completely separate daughter cells. The mechanism by which midbody abscission is achieved remains unknown.

So far, requirements for membrane fusion events during cytokinesis have been identified only for stages that precede midbody abscission. For example, exocytosis supplies the necessary additional surface area used for furrow ingression during cytokinesis in C. elegans and sea urchin embryos (Jantsch-Plunger and Glotzer, 1999; Shuster and Burgess, 2002). In C. elegans, furrow ingression appears to require Syn-4, a syntaxin family member of the SNARE membrane fusion machinery (Jantsch-Plunger and Glotzer, 1999). The function of Syn-4 may not be restricted to furrow ingression, however, because its disruption also causes defects in nuclear envelope reformation. Rab3, a member of a family of proteins implicated in the regulation of SNARE function, has been implicated in furrow ingression in sea urchin embryos but it may also act at earlier stages because its disruption also leads to failure of nuclear division (Conner and Wessel, 2000). SNARE proteins such as syntaxin have been implicated previously in cell division in sea urchin embryos.

Cell division is a highly coordinated event requiring a variety of membrane fusion and fragmentation events. During mitosis in higher eukaryotes, for example, the nuclear envelope breaks down into nuclear membrane vesicles after chromosome condensation, and large cytoplasmic organelles such as the Golgi and endoplasmic reticulum (ER) are also believed to fragment. These fragmented organelle membranes then distribute equally into daughter cells and must refuse with each other to reconstitute their respective organelles. In addition to the breakdown and reformation of the nuclear envelope, Golgi, and ER during the cell cycle, the cell also increases its membrane surface area during cell division. The final step in cell division (cytokinesis) in mammalian cells is the cleavage of a narrow cytoplasmic bridge between the two daughter cells, the so called midbody.

Interestingly, recent experiments in C. elegans have indicated that midbody abscission can be inhibited using the fungal metabolite brefeldin A—a drug that disrupts several organelles and trafficking pathways—under conditions that do not affect furrow ingression (Skop et al., 2001). This suggested that different cellular machineries may control fusion events that facilitate furrow ingression and midbody abscission.

### SUMMARY

Two members of the SNARE membrane fusion machinery, syntaxin 2 and endobrevin/VAMP-8, have been identified as being important to cytokinesis in mammalian cells. In embodiments of the present invention, functional inhibition of these proteins causes failure of midbody abscission while earlier steps of cytokinesis are unaffected. These results indicate that the terminal step of cytokinesis is not a passive “ripping-apart” or “pinching-off” mechanism but is regulated by a SNARE-mediated membrane fusion event which is distinct from exocytic events that are involved in prior ingression of the plasma membrane.

The terminal step of cytokinesis in animal cells is the abscission of the midbody, a cytoplasmic bridge that connects the two prospective daughter cells. Two members of the SNARE membrane fusion machinery, syntaxin 2 and endobrevin/VAMP-8 appear near the midbody during cytokinesis in mammalian cells. Inhibition of syntaxin 2 and endobrevin/VAMP-8 function by over-expression of non-membrane anchored mutants of these proteins causes failure of cytokinesis leading to the formation of binucleated cells. Time-lapse microscopy shows that only midbody abscission but not further upstream events, such as furrowing, are affected. These results indicate that successful completion of cytokinesis requires a SNARE-mediated membrane fusion event, and that this requirement is distinct from exocytic events that may be involved in prior ingression of the plasma membrane.

The present invention is based, at least in part, on the discovery that SNARE proteins are involved in cytokinesis and interference of the SNARE proteins mechanism inhibits cytokinesis resulting in the formation of binucleated cells, and finally, apoptosis of the binucleated cells. An embodiment of the present invention involves the new uses of the SNARE protein mechanism for use in screening for anti-tumor agents. It has not been known in the art to use the SNARE protein mechanism in a screening assay for agents for use as anti-tumor compounds.

A non-limiting embodiment of the invention involves a method of inhibiting uncontrolled cell growth in a population of cells or a tissue comprising contacting said cells with an effective amount of an inhibitor agent of SNARE proteins to cause formation of binucleated cells in the population and eventual apoptosis of the cells. Such agents can include, but are not limited to, natural products, DNA constructs, toxins, antibodies, or competing analogs of SNARE proteins.

A further non-limiting embodiment of the invention involves a method of inducing apoptosis in tumor cells comprising contacting said tumor cells with an effective amount of an agent that inhibits SNARE proteins and their function. Such agents can include, but are not limited to, natural products, DNA constructs, toxins, antibodies, or competing analogs of SNARE proteins.

A further non-limiting embodiment of the invention involves a method of identifying an agent that inhibits uncontrolled cell growth by interfering with the SNARE protein mechanism comprising contacting a sample of said cells with an agent to be tested that inhibits SNARE proteins and their function; monitoring the cell sample or tissue for prevention or inhibition of cytokinesis; whereby prevention or inhibition of cytokinesis in said sample is indicative of an agent that inhibits uncontrolled cell growth.

A further non-limiting embodiment of the invention involves a method of identifying an agent that induces apoptosis in one or more tumor cells, the method comprising contacting a sample of the cells with an agent to be tested; monitoring the cells in the sample for: prevention or inhibition of cytokinesis, formation of binucleated cells, and or cell apoptosis; whereby prevention of cytokinesis, the formation of binucleated cells, and or cell apoptosis in the sample is indicative of an agent that induces apoptosis.

A further non-limiting embodiment of the invention involves a composition comprising an agent that inhibits SNARE proteins induces apoptosis in cells, and an acceptable carrier. Alternatively, the composition may comprise an agent that inhibits SNARE proteins and inhibits uncontrolled cell division or growth, and an acceptable carrier.

A further non-limiting embodiment of the invention involves a method of stimulating cell growth comprising introducing nucleic acid encoding a SNARE protein whereby said nucleic acid is expressed. Alternately, a composition comprising an agent that enhances SNARE protein expression may be useful in stimulating cell growth.

The present invention further relates to the use SNARE protein antibodies or to the use of anti-sense SNARE protein compounds for use as anti-tumor agents. Such agents may be used in combination with existing and new treatment therapies, such as drugs and radiation, which induce apoptosis in tumor cells or which increases the sensitivity of tumor cells to these therapy modalities.

Embodiments of the present invention further comprise a method of inhibiting tissue growth comprising: administering to cells of said tissue an effective midbody abscission inhibiting amount of a composition comprising a SNARE dominant negative inhibitor and a pharmaceutically acceptable carrier. The tissue may be a neoplasm, sarcoma, carcinoma, neural sarcoma, leukemia, lymphoma, and combinations of these. The method may further include a treatment such as chemotherapeutics, radiation, homeopathics, and combinations of these.

Another embodiment of the present invention is a method of identifying compositions that will be useful in preventing midbody cleavage in a sample of cells during cell division including determining whether the composition prevents midbody cleavage during cell division by the number binucleated cells formed in the sample; an increased number of binucleated cells in the sample being an indication that the composition prevents midbody cleavage during cell division. This method may further include controlling cell division in a sample of cells comprising; administering a therapeutically effective amount of a SNARE inhibiting composition which was identified above to the sample of cells in need thereof to inhibit midbody cleavage of said cells; the composition for preventing midbody clevage in cells may include expressing a functionally inhibiting SNARE isoform with an adenovirus vector with a tetracycline-regulatable promotor or by transient transfection of plasmid DNA. The method may involve a sample of cells or tissue which include cells from a neoplasm.

In part, other aspects, features, benefits and advantages of the embodiments of the present invention will be apparent with regard to the following description, appended claims and accompanying drawings where:

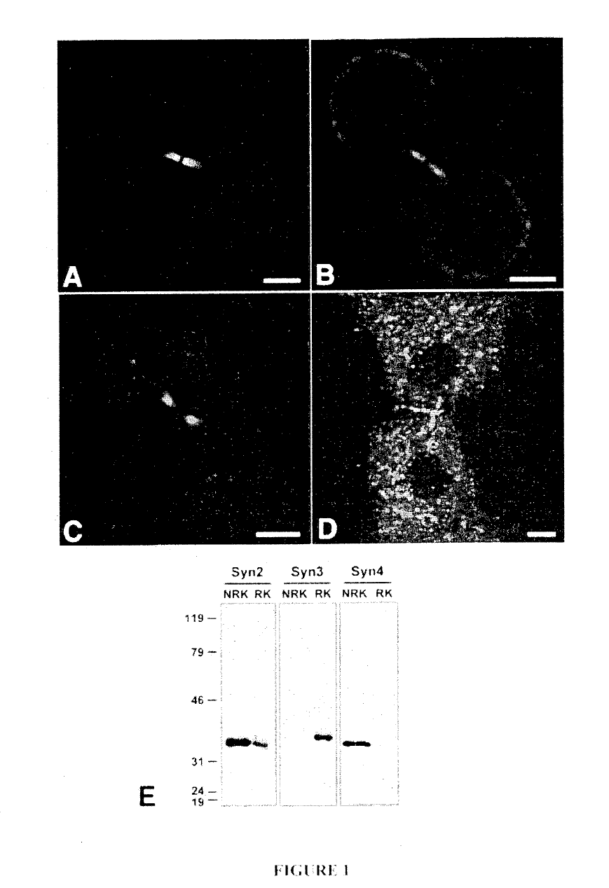

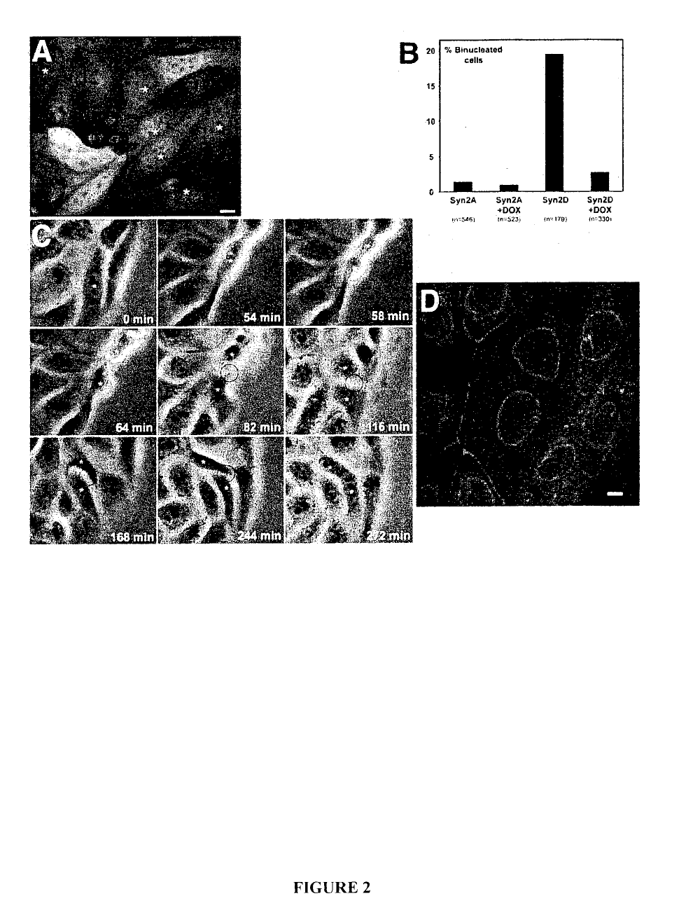

FIG. 1: Syntaxin 2 localizes to the midbody during cytokinesis. (A) Co-immuno-localization of syntaxin 2 (green) and β-tubulin (red) in NRK cells during the midbody-stage of cytokinesis. Nuclei are stained with DAPI (blue). (B) Competition with purified antigen eliminates syntaxin 2-specific staining. Neither syntaxin 3 (C) nor syntaxin 4 (D) localize to the midbody of dividing cells. Scale bars, 5 μm. (E) Immunoblot analysis of NRK cell lysates shows that syntaxins 2 and 4 are abundantly expressed in NRK cells whereas the expression level of syntaxin 3 is relatively low. Equal amounts (15 μg protein) of total rat kidney (RK) lysates were used as controls. Molecular weight makers are indicated in kDa;

FIG. 2: Expression of soluble syntaxin 2 inhibits midbody abscission resulting in binucleated cells. (A) Syntaxin 2D, a splice isoform lacking a transmembrane anchor, was expressed in MDCK cells. Immuno-staining for syntaxin 2 (green) reveals expressing cells. Binucleated cells are denoted by asterisks; scale bar, 5 μm. (B) Binucleated cells formed after 16 hour expression of syntaxin 2D were subjected to double-immunostaining for the Nuclear Transport Factor p97 (green) and the tight junction protein ZO-1 (red). Note that the nuclei of binucleated cells exhibited normal p97-staining indicating that nuclear division and reformation of the nuclear envelopes was unaffected. (C) Frames of time-lapse phase contrast microscopy of MDCK cells expressing syntaxin 2D. For orientation, the cell if interest is highlighted by asterisks and the midbody is circled. Quantification of failed cytokinesis after 16 hour expression of the full-length syntaxin 2A or the truncated syntaxin 2D using adenovirus vectors calculated as the fraction of nuclei in binucleated cells as the percentage of the total nuclei. As negative controls, syntaxin expression was prevented by the addition of doxycycline (+DOX). The total numbers of nuclei counted for each condition are indicated. (E) Failed cytokinesis after 24 hour expression of syntaxin 2A, syntaxin 2D, or truncated versions of syntaxins 3 or 4 lacking transmembrane anchors by plasmid-mediated transient transfection.

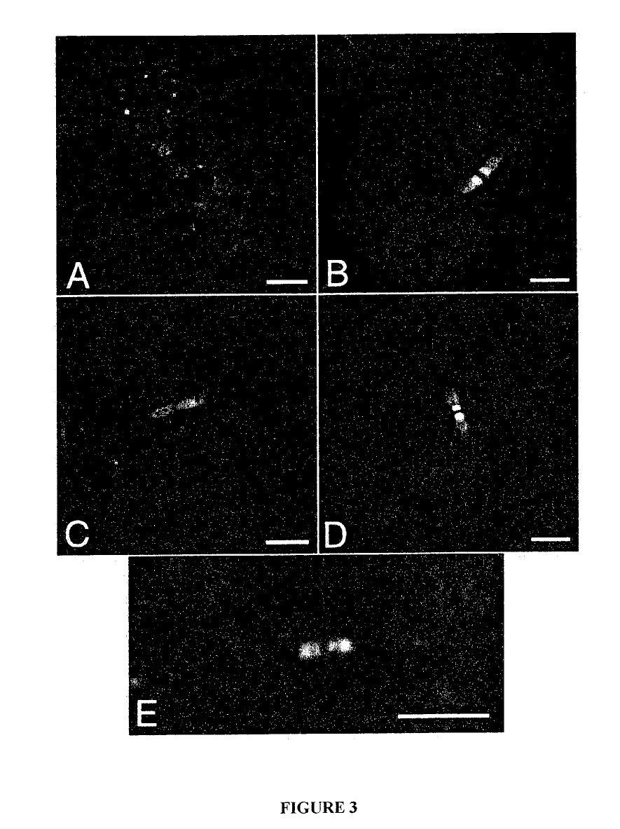

FIG. 3: Endobrevin co-localizes with syntaxin 2 on the midbody. (A) Cellubrevin (green) localizes to intracellular vesicles in late telophase NRK cells but not to the midbody which is identified by immuno-staining for P-tubulin (red). (B) Endobrevin (green) localizes to midbody in NRK cells. The immuno-staining for endobrevin is eliminated by competition with total bacterial lysate containing GST-endobrevin (C) but not by lysate containing GST (D). (E) Co-immunostaining for syntaxin 2 (green) and endobrevin (red) reveals their co-localization on the midbody of NRK cells. Scale bars, 5 μm;

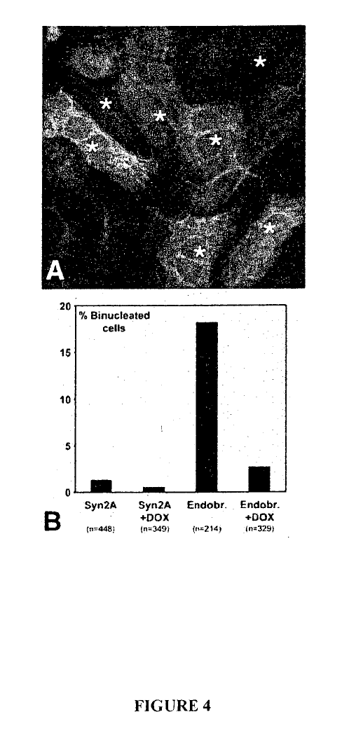

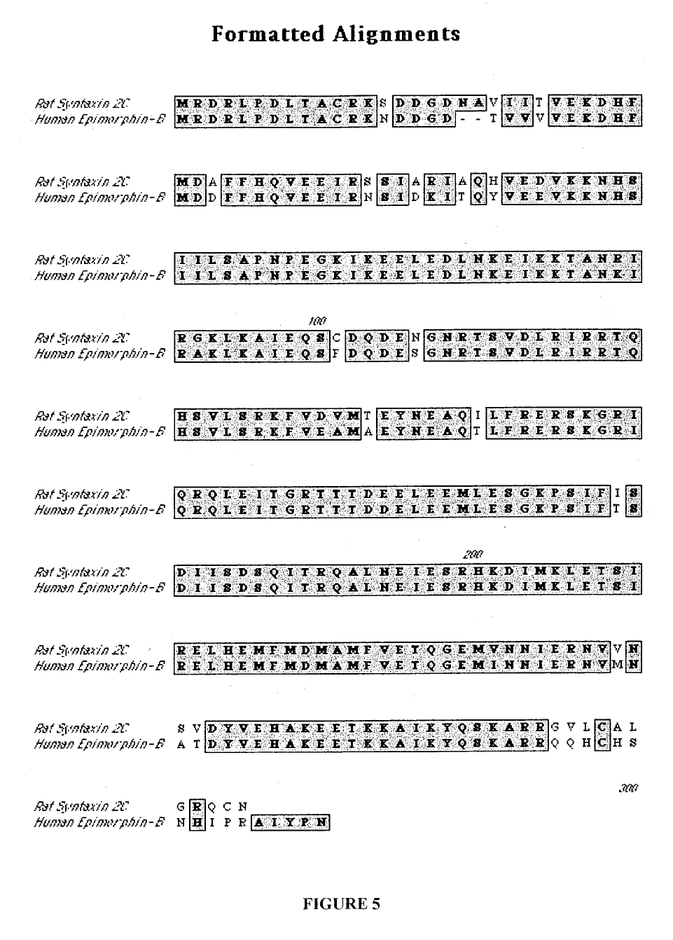

FIG. 4: Endobrevin function in cytokinesis. (A) Expression of truncated endobrevin lacking its transmembrane anchor (green) in MDCK cells for 16 hours results in the formation of binucleated cells (denoted by asterisks). (B) Quantification of failed cytokinesis after expression of truncated endobrevin or syntaxin 2A as a control for 16 hours. As a further control, expression was suppressed by doxycycline (DOX);

FIG. 5: Formated alignments of (SEQ ID NO:11) Rat Syntaxin 2C and (SEQ ID NO: 13) Human Epimorphin-B;

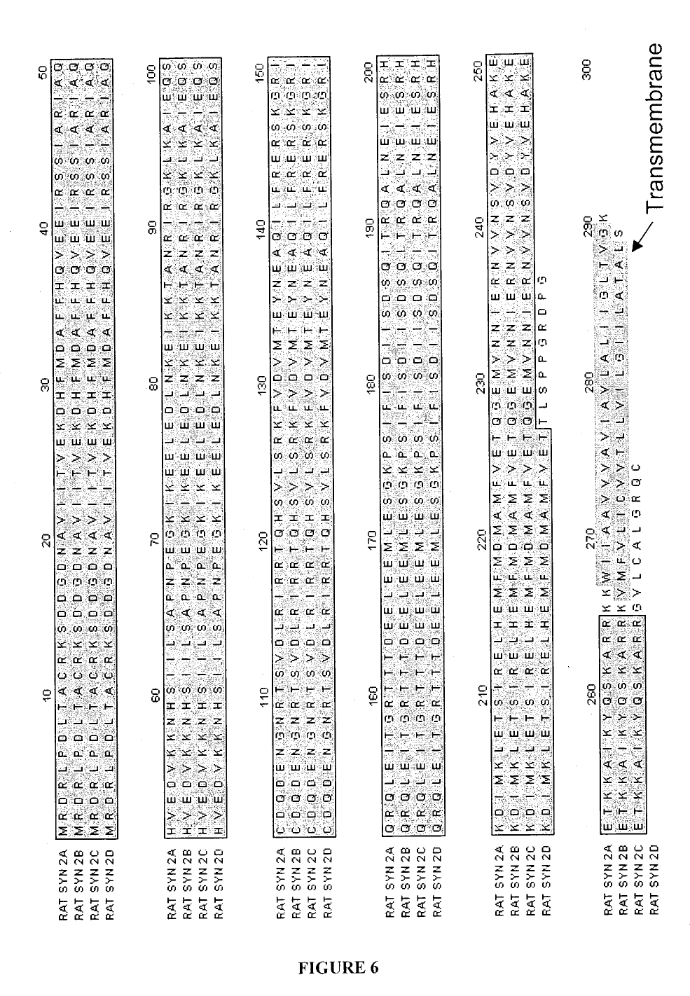

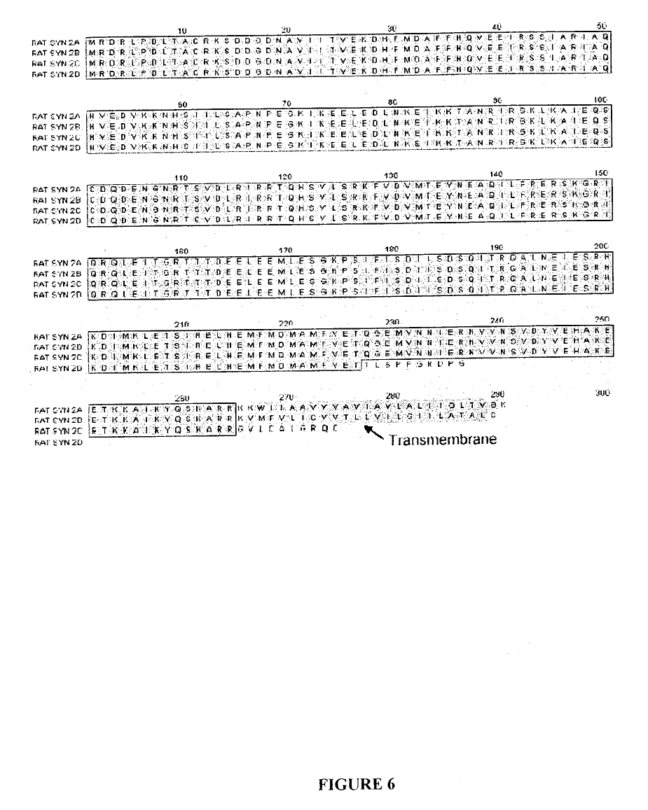

FIG. 6: Alignment of protein sequences of four known (SEQ ID NO:9-13) Syntaxin 2 splice-isoforms in rat.

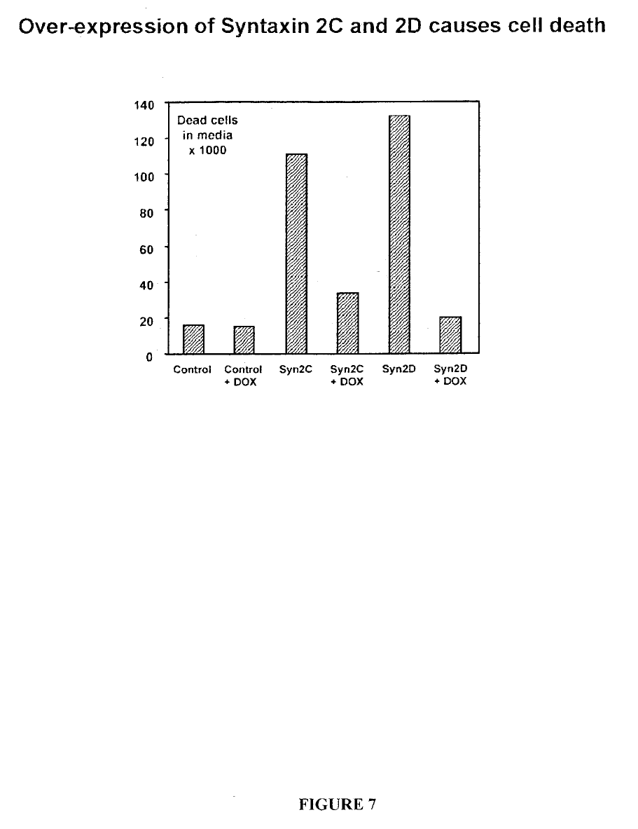

FIG. 7: Over expression of Syntaxin 2C and Syntaxin 2D in MDCK cells causes cell death;

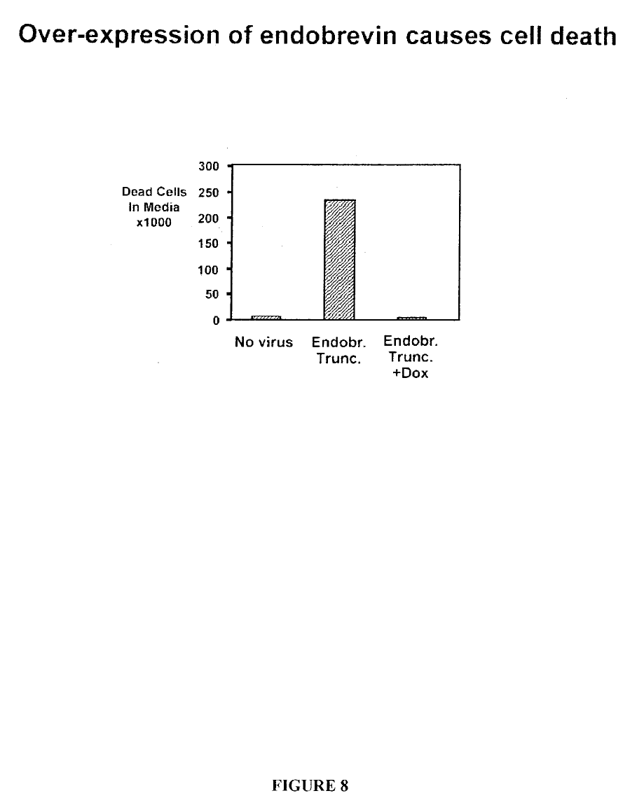

FIG. 8: Over expression of Endobrevin in MDCK cells causes cell death.

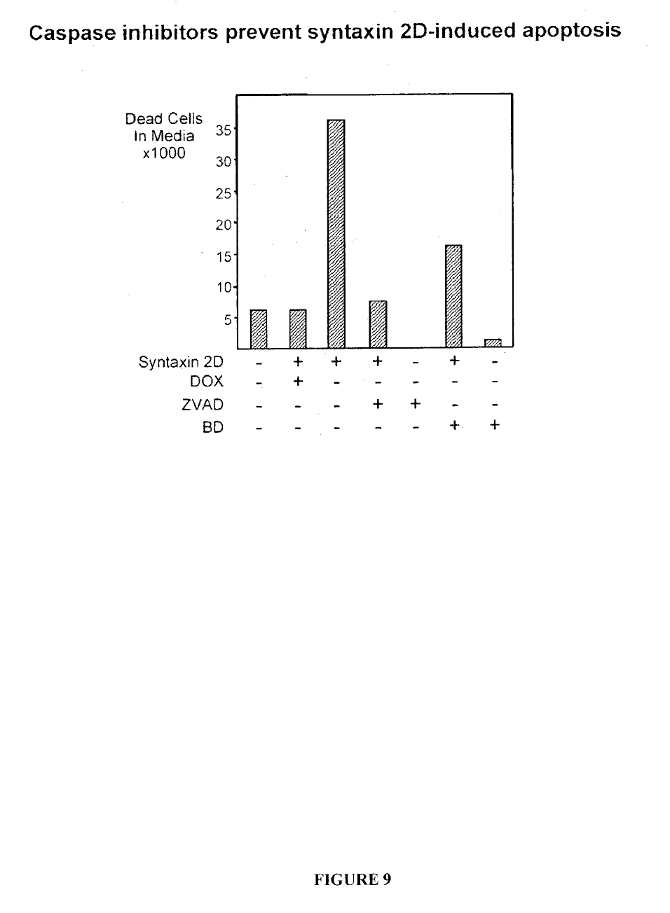

FIG. 9: Caspase inhibitors prevent syntaxin 2D-induced apoptosis.

### DETAILED DESCRIPTION OF THE INVENTION

A highly conserved set of membrane proteins has been identified that are involved in many types of intracellular fusion. These proteins localize to both vesicle and target membranes, and are known as soluble NSF attachment protein receptors (SNAREs); NSF stands for N-ethyl-maleimide-sensitive fusion protein. SNAREs appear to function throughout the secretory pathway as the minimal machinery driving membrane fusion. SNAREs may be conveniently divided into v-SNAREs and t-SNAREs depending upon their localization to a vesicle or a target membrane in a cell fusion event.

Proteins in the SNARE superfamily include SNAP-25, syntaxin (also known as t-SNARE), and the vesicle-associated membrane protein (VAMP, also known as v-SNARE). These SNAREs were identified in the sea urchin egg in association with cortical granules, secretory vesicles whose contents give rise to the fertilization envelope. Syntaxin and VAMP are also present throughout embryogenesis enriched in cells with elevated levels of regulated secretion. During the cleavage stage of this embryo, a period of cell division every 45-60 min, there is enrichment of these molecules on vesicles accumulating at the cortex of cells, suggesting that these vesicles may play an important role in cell division. Thus, it is likely that these proteins not only mediate the complex array of membrane fusion events of secretion, as previously documented, but also function in the contribution of new membrane to the cell surface during division.

While not wishing to be bound by theory, it appears midbody cleavage during cell division, cytokinesis is accomplished by a SNARE-mediated mechanism in mammalian cells. The SNARE-mediated mechanism involves, but is not limited to, syntaxin 2 (also known as epimorphin) and endobrevin (also known as VAMP-8). In embodiments of the present invention, functional control of SNAREs have been demonstrated to regulate midbody abscission. Regulation may refer to either prevention or stimulation of cell growth, division, or cell apoptosis. In preferred embodiments inhibition of SNAREs results in the formation of binucleated cells. Method of identifying the location of such SNAREs, for example (SEQ ID NO: 1-13) in the midbodies of cells are disclosed as are methods of expressing and identifying compositions which inhibit the functionality of these proteins.

Typically, endogenous expression levels of SNAREs are often low which makes detection challenging. The localization of SNAREs may be determined in cells by immunofluorescence microscopy including methods for signal amplification, antigen retrieval and suppression of antibody cross-reactivity. To define which trafficking pathway a SNARE of interest is involved in, one needs to specifically inhibit its function. One technique to accomplish this is to introduce inhibitors of SNARE function—such as antibodies or clostridial toxins—into cells by plasma membrane permeabilization using the bacterial toxin streptolysin-O or microinjection. Using antibodies against syntaxin 2 and endobrevin SNAREs, the antibodies appear to localize to the region of the midbody during cytokinesis.

Midbody-cleavage may be prevented by interfering with the function of either syntaxin 2 or endobrevin in these cells. Interestingly, inhibition of syntaxin 2 or endobrevin function does not interfere with midbody-formation, but does prevent midbody cleavage. Inhibition of midbody-cleavage eventually leads to binucleated cells and subsequent cell death or apoptosis. Over-expression of syntaxin 2 isoforms that lack a C-terminal hydrophobic domain inhibit or prevent cytokinesis. Similarly, over-expression of a C-terminally truncated version of endobrevin inhibit or prevent cytokinesis. These manipulations of syntaxin 2 lead to the formation of binucleated cells as shown by fluorescence microscopy and by time-lapse microscopy. Binucleated cells that have formed due to failure of midbody-cleavage eventually apoptose. This involves caspases. Caspases inhibitors, such as but not limited to ZVAD and BD may be used with functional inhibitors of syntaxins, like syntaxin 2D, to control apoptosis in a sample of cells as illustrated in FIG. 9.

Therefore, the methods and compositions to interfere with a SNARE-mediated mechanism and prevent cytokinesis thereby inhibiting cell division include, but are not limited to, inhibition of SNAREs during cytokinesis, inhibition of SNAREs such as syntaxin 2 and or endobrevin in cells during cytokinesis, and inhibition of SNAREs such as syntaxin 2 and or endobrevin localized to the midbody of cells during cytokinesis. Preferably the methods and compositions of the present invention inhibit or prevent syntaxin 2 or endobrevin function through the action of over-expressed of syntaxin 2 or endobrevin isoforms. Although methods to interfere with a SNARE-mediated mechanism and prevent cytokinesis thereby inhibiting cell division have numerous applications, one specific application is for use in cancer therapy to inhibit cell division. Existing methods often have side effects on non-tumor cells. In theory, the inhibition of midbody cleavage using this technique should only affect rapidly dividing cells and may be more specific than existing methods. Methods can be devised to express the inhibitors of the identified SNARE proteins in tumor cells, e.g. by viral gene transfer. This method may prove useful in the treatment of cancers.

The sequence listing for cDNA encoding human epimorphin (syntaxin 2), SEQ ID NO: 1, and cDNA encoding the B isoform of human epimorphin, SEQ ID NO: 2, have been reported (Hirai et al 1994). The sequence listing for homo sapiens vesicle-associated membrane protein 8 (endobrevin) (VAMP8), mRNA, SEQ ID NO: 3, have also been reported (Wong et al 1998). The methods of the present invention may be used to prepare compositions comprising truncated non-membrane anchored or dominant negative inhibitors of SNAREs using various vectors, or regulated vector systems for expressing them in both human, and rat, and other mammals. The methods of embodiments of the present invention may be used to determine whether SNAREs such as human empimorphin (SEQ ID NO:1) or human endobrevin (SEQ ID NO:3) localize to the midbody of cells in a sample of cells or tissue during cytokinesis. The methods of various embodiments of the present invention may also be used to identify whether the known or unknown truncated human isoform inhibitors of these human SNAREs are effective in forming non-functional complexes with SNAREs in cells or SNAREs localized to the midbody of such cells. Such non-functional SNARE complexes may form binucleated cells, prevent cytokinesis, and or causing cell apoptosis. Similarly, in accordance with the methods described herein, one may identify compounds or drug formulations that mimic the effect of the truncated forms of syntaxin 2.

The present invention relates to the use of inhibitors of SNARE proteins in the prevention of cytokinesis to prevent uncontrolled cell growth and division. A method of preventing cell division in a collection of cells may include administering to a sample of cells or tissue an effective amount of a composition inhibiting SNARE function during cytokinesis in the cells. The methods and composition of the present invention may also be used as an assay screen for other related inhibitors or agents that prevent cytokinesis. This embodiment of the present invention relates to identification of SNAREs within cells including those localized in the midbody region of dividing cells, the expression of inhibitors to these SNAREs, and identification of inhibitors of SNAREs in preventing cell division based on the formation of binucleated cells or prevention of midbody abscission. Such methods and compositions may be used with cells in benign or malignant neoplasia and may include tumors such as but not limited to sarcomas, carcinomas, lymphomas, leukemias, or neoplasms of the nervous system.

Syntaxin 2 appears to localize to the midbody. Membrane fusion events in intracellular vesicle trafficking pathways are generally mediated by proteins of the SNARE super-family which consists of several sub-families including syntaxins (Chen and Scheller, 2001; Jahn and Sudhof, 1999; Weimbs, et al., 1997). Analysis of the localization of syntaxin 2 in cultured proliferating NRK (normal rat kidney) cells led to the following serendipitous finding. An affinity-purified antibody against syntaxin 2 strongly labeled small structures in a fraction of the cell population. Co-labeling for β-tubulin identified these structures as midbodies. Syntaxin 2 immunoreactivity localized to distinct regions of ˜1 μm apparent diameter on either side of the midbody (FIG. 1A). These syntaxin 2 regions were intersected by microtubules. This staining pattern was consistently observed using two independently raised syntaxin 2 antibodies and could be eliminated by competing antigen indicating that it is specific as illustrated in FIG. 1B. Identical staining patterns were also observed with several other mammalian cell lines including (Chinese Hamster Ovary) CHO cells and human HEK293 cells (not shown) and illustrates that this technique may be applied to other syntaxins and cells to identify SNARE type and their location in dividing cells.

Syntaxin 2 is a ubiquitously expressed t-SNARE (Bennett et al., 1993) that has been reported to be targeted to the plasma membrane in several cell types including polarized Madin Darby canine kidney (MDCK) cells (Li et al., 2002; Low et al., 1996; Low et al., 2000). Two other widely expressed plasma membrane t-SNAREs are syntaxins 3 and 4 (Li et al., 2002; Low et al., 1996). Western blot analysis showed that NRK cells express all three syntaxins (FIG. 1E). However, neither syntaxin 3 nor syntaxin 4 exhibited the same midbody localization as syntaxin 2 during cytokinesis (FIG. 1C, D).

The subcellular steady-state location of a given t-SNAREs generally corresponds to the site at which the t-SNARE functions. The localization of syntaxin 2 at the midbody therefore suggested that it may be involved in a fusion event during cytokinesis. Syntaxin 2 may be involved in increasing the cell surface area during furrowing by mediating the fusion of vesicles with the plasma membrane close to the site of ingression. Syntaxin 2 may be directly involved in the final abscission of the midbody to result in completely separated daughter cells.

Syntaxin 2 function during cytokinesis. To investigate what function syntaxin 2 plays in cytokinesis and to examine its mechanism of action a dominant-negative approach was employed. The over-all domain structure of syntaxins is highly conserved (Weimbs, et al., 1997; Weimbs, et al., 1998), and they are characterized by a C-terminal transmembrane anchor while the rest of the molecule protrudes into the cytoplasm. Recombinant soluble SNAREs that lack their membrane anchors are known to inhibit membrane fusion by forming nonfunctional complexes with endogenous SNARE proteins (Hua and Scheller, 2001). A brain-specific, alternatively spliced isoform of syntaxin 2, termed syntaxin 2D, has previously been identified that lacks a transmembrane anchor, it is a truncated mutant, while the remainder of the cytoplasmic domain is identical to full-length syntaxin 2 (Quinones et al., 1999). The function of syntaxin 2D is unknown. However, it was reported to be a soluble cytoplasmic protein (Quinones et al., 1999) and would be predicted to act as a dominant-negative inhibitor of the function of membrane-anchored syntaxin 2.

In an embodiment of the present invention, syntaxin 2D, rat, was expressed in MDCK cells using an adenovirus vector with a tetracycline-regulatable promotor. Other virus vectors and regulators may be used as would be obvious to try by those skilled in the art. Immunofluorescence analysis confirmed the cytoplasmic localization of syntaxin 2D (FIG. 2A). Syntaxin 2D expression for 16 hours resulted in a high frequency of binucleated cells indicating that the cells had undergone nuclear division in the absence of cytokinesis (FIG. 2A, B). Similarly, it was observed that overexpression of syntaxin 2C resulted in an increase in the fraction of binucleated cells formed in MDCK cells. This effect could be prevented by suppressing syntaxin 2C or 2D, FIG. 2 and FIG. 7, expression by the addition of doxycycline indicating that the observed block of cytokinesis is not due to the adenoviral infection. Furthermore, adenovirus-mediated expression of the membrane-anchored, full-length syntaxin 2A did not result in an increase in binucleated cells (FIG. 2B). These results indicated that syntaxin 2 function is involved in cytokinesis and that composition inhibiting SNARE function during cytokinesis or mid-body abscission will cause binucleated cells to form. Such compositions inhibiting SNARE function may be used to control or prevent cytokinesis or midbody abscission. Given that only ˜50% of the cells underwent mitosis during the course of these experiments it is estimated that cytokinesis failed in approximately 60% of the mitotic events in cells that expressed syntaxin 2D. The expression of this isoform may form the basis of a method of preventing mid-body cleavage in cells which comprises administering to cells an effective amount of a composition inhibiting mid-body cleavage of the cells. Further, the immunofluorescence and counting of cells serve as an assay for determining whether any expressed isoform is functional for inhibiting midbody cell abscission.

As a further control for the specificity of the dominant-negative inhibition of syntaxin 2 function, truncated versions of syntaxins 3 and 4—lacking the transmembrane anchors—were expressed in MDCK cells by transient transfection of plasmid vectors. This was compared to transient transfection of syntaxin 2A or 2D cDNAs inserted into identical plasmid vectors. Similar to the adenoviral gene transfer above, expression of syntaxin 2D for 24 hours resulted in a high frequency of binucleated cells (FIG. 2E). In contrast, neither expression of the membrane-anchored syntaxin 2A nor of the truncated syntaxins 3 or 4 (human) had this effect. This result indicates that the dominant-negative inhibition by non-membrane anchored syntaxins is specific, and that syntaxin 2 is specifically involved in cytokinesis.

Syntaxin 2 function during midbody abscission. To distinguish whether syntaxin 2 inhibition prevents the ingression of the cleavage furrow or the abscission of the midbody, syntaxin 2D-expressing cells were investigated by time-lapse microscopy. FIG. 2C shows representative frames. In 6 independent time-lapse experiments, 41 events were observed that resulted in the formation of binucleated cells. In all cases, nuclear division, cleavage furrow formation and ingression, and the formation of midbodies were indistinguishable from controls. However, the cells were unable to undergo midbody abscission. The average time that the syntaxin 2D-expressing cells remained in the midbody stage was 153 min (range 64-355 min, n=41) after which midbody regression occurred to lead to binucleated cells. Syntaxin 2D or cells treated to express syntaxin 2D may be used to form a composition comprising which inhibits mid-body abscission in cells. This method may be used to identify compositions that will inhibit/prevent midbody cleavage during cell division and comprises determining whether the composition inhibits or prevent midbody cleavage during cell division by the number binucleated cells formed in the treated sample; an increased number of binucleated cells being an indication that the composition inhibits or prevents midbody cleavage during cell division. The result of this test may be used to identify compositions that prevent midbody cell abscission in a cell population, and such compositions may then be administered in a therapeutically effective amount to cells in need of prevention or inhibition of midbody abscission.

To investigate whether syntaxin 2 inhibition might affect the reassembly of the nuclear envelope, the binucleated cells were immuno-stained with antibodies against the Nuclear Transport Factor p97 (FIG. 2D) or lamin B2 (not shown). The nuclear envelopes of the binucleated cells appeared to be complete and intact and were indistinguishable from those of control cells indicating that syntaxin 2-inhibition has no effect on the nuclear envelope and that nuclear division had occurred unperturbed. Evidence for micronuclei or nuclear buds in the binucleated cells after syntaxin 2-inhibition was not observed. These defects would be indicators of loss or malsegregation of chromosomes as a result of defects in the spindle, centromeres or as a consequence of chromosome undercondensation (Fenech and Crott, 2002). Overall, these results indicate that syntaxin 2 functions during midbody-abscission but its inhibition does not appear to affect further upstream events of mitosis such as chromosome segregation, nuclear envelope reassembly, furrowing etc. Furthermore, the results indicate that midbody-abscission involves a SNARE-mediated fusion event, and suggest that this event requires a different fusion machinery than exocytosis for the delivery of new membrane to aid in furrow ingression. Finally, if midbody abscission is blocked by syntaxin 2-inhibition, cells can not otherwise “rip apart” or “pinch off” to complete cytokinesis.

Endobrevin/VAMP-8 functions together with syntaxin 2 during midbody abscission. If syntaxin 2 mediates a membrane fusion event that severs the midbody, it would be predicted to involve other members of the SNARE machinery as well. In other intracellular fusion events, small v-SNAREs of the synaptobrevin/VAMP family mediate membrane fusion in concert with syntaxins. A v-SNARE involved in cytokinesis would be expected to exhibit a relatively ubiquitous tissue expression pattern. Experiments were performed to investigate whether the two ubiquitously expressed v-SNAREs cellubrevin/VAMP-3 or endobrevin/VAMP-8 localize to the midbody region during cytokinesis. FIG. 3A shows that cellubrevin is only found on intracellular vesicles but not at the midbody during cytokinesis. In contrast, endobrevin appears to be highly concentrated at the midbody in a staining pattern very similar to that of syntaxin 2 (FIG. 3B, D). Again, the immuno-signal could be eliminated by competition with antigen (FIG. 3C), and two independent endobrevin antibodies resulted in identical staining patterns (not shown). Double-immunofluorescence microscopy with antibodies against syntaxin 2 and endobrevin revealed nearly completely overlapping localizations (FIG. 3E).

To investigate whether endobrevin is functionally involved in cytokinesis, an isoform or truncated mutant of endobrevin, human, lacking the C-terminal transmembrane anchor was prepared and was expressed using a tetracycline-regulatable adenoviral vector as described above for syntaxin 2D. FIG. 4A shows that expressed truncated endobrevin distributes throughout the cytoplasm and results in a high percentage of binucleated cells after 16 hours. As a control, when the expression of truncated endobrevin was prevented by the inclusion of doxycycline, the formation of binucleated cells was suppressed (FIG. 4B). These results indicate that truncated endobrevin acts as a dominant-negative inhibitor of membrane-anchored endobrevin resulting in inhibition of cytokinesis. These results indicated that endobrevin function is involved in cytokinesis and that composition inhibiting SNARE function during cytokinesis or mid-body abscission will cause binucleated cells to form. Such compositions inhibiting SNARE function may be used to control or prevent cytokinesis or midbody abscission. Time-lapse microscopy revealed that endobrevin inhibition did not interfere with events upstream of midbody-formation but resulted in the inability to cleave the midbodies. The average time between midbody-formation and regression into binucleated cells was 173 min (range 80-345 minutes, n=45). This phenotype was indistinguishable from that of syntaxin 2-inhibition as described above, suggesting that syntaxin 2 and endobrevin may act together at the same step during midbody abscission.

Collectively, these results show for the first time that midbody abscission involves the action of members of the SNARE membrane fusion machinery. Inhibition of syntaxin 2 or endobrevin had no apparent effect on cleavage furrow invagination, nuclear division, reformation of the nuclear envelope or other mitotic events. It is therefore unlikely that these SNAREs are involved in any step prior to midbody abscission. Since cleavage furrow invagination is believed to require exocytosis for the insertion of additional plasma membrane it is likely that other SNAREs are involved in this process in mammalian cells. A possible candidate is syntaxin 4 which has been found to localize to the ingressed plasma membranes separating the prospective daughter cells prior to midbody abscission (see FIG. 1D). This would be analogous to the proposed function of Syn-4 in C. elegans (Jantsch-Plunger and Glotzer, 1999). Note that mammalian and C. elegans SNAREs are too divergent to allow assignment of orthologues by sequence comparison (Jantsch-Plunger and Glotzer, 1999; Weimbs, et al., 1997). Therefore, despite the coincidental similarity of their names, it remains to be established whether the mammalian syntaxin 4 may have the equivalent role of the C. elegans Syn-4 in cleavage furrow ingression.

Results of embodiments of this invention show that syntaxin 2 and endobrevin appear to function in midbody abscission during cell division. Further, embodiments of the present invention provide methods for identifying the location of SNAREs in cells undergoing cytokinesis, methods for making and delivering isoforms composition of such SNAREs, and methods for identifying whether cells treated with such SNARE isoforms result in the formation of binucleated cell. The methods and compositions of the present invention may be applied to mammalian cells including human cells for which SNARE proteins and DNA for these proteins are known (SEQ ID NO: 1-3, SEQ ID NO: 13). The terminal step of cytokinesis utilizes a SNARE machinery or function that is distinct from those involved in prior steps that require membrane fusion such as furrowing. If the function of syntaxin 2 or endobrevin is inhibited by SNARE isoforms or other proteins which inhibit their normal function, cell division can not be completed indicating that other SNAREs can not substitute their function. This may mean that midbody abscission is a highly regulated, active process, and that mammalian cells may possess no alternative mechanisms that can accomplish the breakage of this narrow bridge. These results may be used in a method of preventing mid-body cleavage in cells comprising administering to cells an effective amount of a composition inhibiting the function of such SNAREs in mid-body cleavage or abscission of cells.

Cell division is not only a fundamental biological process but is also of particular interest as a target for anti-tumor strategies. Currently used anti-tumor compounds target the cell cycle at various steps. The identification of molecules involved in the terminal step of cytokinesis may provide potential new targets that may be exploited for cancer therapy.

Affinity-purified antibodies against the cytoplasmic domains of rat syntaxins 2, 3 and 4 have been described previously (Low et al., 2000). An antibody against the cytoplasmic, domain of endobrevin was raised and affinity-purified equivalently as described previously (Li et al., 2002). As confirmatory controls, independently raised affinity-purified antibodies against syntaxin 2 (Quinones et al., 1999) and endobrevin (gift from Wanjin Hong, IMCB, Singapore) were used. A monoclonal β-tubulin antibody developed by Michael Klymkowsky was obtained from the Developmental Studies Hybridoma Bank, The University of Iowa. Antibodies against the Nuclear Transport Factor p97 and ZO-1 were from ABR (Golden, Colo.) and Chemicon (Temecula, Calif.), respectively.

Cell culture and immuno-localization; NRK cells (from ATCC) were cultured in DMEM with sodium pyruvate, 10% FBS and penicillin and streptomycin. MDCK cells were cultured as described (Low et al., 2000). Cells were fixed in methanol and subjected to immuno-staining and confocal fluorescence microscopy as described previously (Low et al., 2000). For localizing simultaneously two proteins recognized by rabbit primary antibodies (syntaxin 2 and endobrevin), fluorescein-labeled Fab fragments of the secondary antibody (Jackson ImmunoResearch, West Grove, Pa.) were used after incubation with the first rabbit primary antibody. The cells were briefly fixed again with 4% paraformaldehyde, then incubated with the second rabbit primary antibody, followed by Texas Red-labeled secondary antibody (Weimbs, et al., 2003). Antibody concentrations were titered so that all negative controls were negative.

Expression of SNARE cytoplasmic domains; the adenovirus vectors for tetracycline-regulated expression of rat syntaxins 2A and 2D have been described previously (Quinones et al., 1999). The identical vector system was used for the expression of truncated endobrevin lacking its transmembrane domain. MDCK cells stably expressing the TET-transactivator (Clontech, Palo Alto, Calif.) were infected with virus numbers titered to result in 90% of expressing cells after 16 hours. After fixation, double-immuno-staining for the respective truncated SNARE and the 6.23.3 endogenous plasma membrane marker (Low et al., 2000), and nuclear staining with DAPI, random fields were imaged by fluorescence microscopy, and the number of mono- and bi-nucleated cells were counted manually.

Truncated SNAREs were expressed in MDCK cells as described above. ˜8 hours post-infection, cells were subjected to time-lapse phase contrast microscopy (2 minutes/frame) using a fully motorized Leica DMIRB microscope equipped with a temperature-, CO₂— and humidity-controlled environmental chamber. Images were processed using Metamorph, Adobe Photoshop and Quicktime.

Time-lapse phase contrast microscopy of MDCK cells, FIG. 2C, expressing syntaxin 2D were recorded at 2 minutes/frame and a movie recorded at 10 frames/second. The experiment shows cell with failing cytokinesis due to expression of syntaxin 2D.

Time-lapse phase contrast microscopy of MDCK cells expressing truncated endobrevin were recorded at 2 minutes/frame and a movie recorded at 10 frames/second. The experiment shows cells with failing cytokinesis due to expression of truncated endobrevin.

The endogenous expression levels or most SNAREs are relatively low (with the exception of neuronal SNAREs in neurons) which can make detection a challenge. Below are descriptions of methods for enhancing signals in immunofluorescence experiments using cultured cells or tissue sections. Protein and nucleotide sequences for human and rat syntaxins, endobrevin, and their isoforms are given in Table 1 and in the sequence listing.

TABLE 1

| SEQ ID NO | DESCRIPTION |
|---|---|
| 1 | Human epimorphin cDNA |
| 2 | Isoform B of human epimorphin cDNA |
| 3 | Homo sapiens membrane protein 8 (endobrevin) mRNA |
| 4 | Rattus norvegicus endobrevein mRNA |
| 5 | Rattus norvegicus Syntaxin 2A, Syntaxin 2, mRNA |
| 6 | Rattus norvegicus Syntaxin 2B, Syntaxin 2′, mRNA |
| 7 | Rattus norvegicus Syntaxin 2C, Syntaxin 2″, mRNA |
| 8 | Human epimorphin, mRNA |
| 9 | Rat syntaxin 2A, Syntaxin 2 protein |
| 10 | Rat syntaxin 2B, Syntaxin 2′ protein |
| 11 | Rat syntaxin 2C, Syntaxin 2″ protein |
| 12 | Rat syntaxin 2D protein |
| 13 | Human epimorphin B protein |

Immunofluorescence-staining with signal amplification by anti-fluorescein tertiary antibody. This method uses a primary antibody against a SNARE protein, followed by a fluorescein-labeled donkey anti-rabbit-IgG secondary antibody. To enhance the signals, a rabbit antibody is then used that recognizes the fluorescein and is coupled to the fluorophore Alexa-488 (Molecular Probes Eugene, Oreg., cat# A-11090). Since the spectral properties of Alexa-488 are nearly identical to fluorescein, the result is an amplification of the “FITC” signal.increases of approximately five-fold. The method comprises:

(1) MDCK cells are cultured on 12 mm Transwell filters in MEM containing 10% FBS. The cells are allowed to polarize for at least 3 days, with changes of media every other day; (2) Rinsed cells briefly 3 times with PBS containing 1 mM each CaCl2 and MgCl2 (PBS+); (3) Fix with 4% paraformaldehyde in PBS+ for 20 min at room temperature. During fixation, the cells are placed on an orbital shaker at very low speed (alternatively, cells can be fixed in cold methanol at 20° C. for 10 min); (4) Quench any remaining fixative with 75 nM NH4Cl and 20 mM glycine (both from a 1 M stock) in PBS at RT for 10 min with shaking (in case of the methanol fixation, the quenching step is omitted); (5) After 2 brief rinses in PBS, the cells are blocked and permeabilized in Block Solution (PBS containing 3% BSA and 0.2% TX-100) at 37° C. for 30 min; (6) Primary antibody diluted in Block Solution is centrifuged at 12,000 g for 15 min to pellet any aggregates; (7) The membrane is carefully cut out of the Transwell mount and placed on parafilm on a 30 μl drop of the antibody. Another 30 μl of the diluted antibody is placed on tope of the filter. Incubation is in a humid chamber at 37° C. for 2 hours; (8) the membrane is transferred back to a 12 well dish and washed 4 times 5 min in Wash Solution) PBS with 0.05% TX-100 and 0.7% fish skin gelatin (Sigma Cat # G-7765, St. Louis, Mo.); (9) the secondary antibody conjugated with fluorescein is diluted in Wash Solution and again centrifuged for 15 min to pellet out any aggregates and applied to the membrane as described for the primary antibody. Incubation is for 1 hr at 37° C. in a humid chamber; (10) the membrane is washed 4 times 5 min in Wash Solution and the Alexa 488 conjugated anti-fluorescein is diluted in Wash Solution and applied to the membrane after centrifugation as described previously. The antibody is allowed to incubate for 1 hr at 37° C.; (11) excess antibody is removed by washing 4 times 5 min in Wash Solution and 2 times 3 min in PBS containing 0.1% TX-100, followed by 2 rinses in PBS; and (12) the membrane is mounted cell side up and is ready for viewing under the microscope.

Antigen Retrieval by pressure-cooking. Immuno-fluorescence staining of SNAREs in sections of fixed tissues often results in weak signals. This may be due to the fact that SNAREs appear to spend most of their time in complexes with other SNAREs or regulatory proteins. Fixation with protein-cross-linking fixatives, like formaldehyde, may then mask many epitopes. This could theoretically lead to localization artifacts because a sub-population of a SNARE may be “invisible” by immuno-staining. Several methods are used to unmask epitopes in tissue sections, including digestion with proteolytic enzymes, denaturation with urea, SDS, or guanidine hydrochloride, and heat-treatment. Preferably heat-treatment using a pressure cooker leads to the most reproducible signal enhancement while preserving tissue morphology. Paraffin sections of tissues from animals perfusion-fixed with 4% paraformaldehyde in PBS+ have worked well. The method comprises:

(1) Deparaffinize tissue and re-hydrate sections on slides as usual (leave slides wet until ready for pressure-cooking); (2) make 2 liters of 10 mM Na-citrate buffer, pH 6.0 by dilution from 1 M stock; (3) put citrate buffer in large stainless-steel household pressure-cooker (must not be aluminum as it reacts with the citrate). With the lid only loosely on the cooker heat until boiling; (4) place slides in a glass or steel slide-holder and put into boiling citrate buffer; (5) close lid tightly. Place the weight on the pressure-cooker's valve; (6) continue to heat until weight starts to wobble; (7) heat for one more minute; (8) remove cooker from heater, place under running cold water tap; (9) once pressure is down, open lid and flood cooker with running cold tap water; (10) take out slides and proceed with immuno-staining as usual.

Suppression of antibody cross-reactivity. Immuno-localization experiments of endogenous proteins suffer from the inherent problem that usually no negative control is available (in contrast to experiments with transfected cells in which the non-transfected cells can serve as the negative control). Omitting the primary antibody only controls for autofluorescence, background by the secondary antibody etc., but it does not establish specificity of the primary antibody. Competition with the antigen is better but does still not exclude cross-reactivity of the primary antibody. Polyclonal antibodies have to be affinity-purified against the antigen. Unpurified antisera almost always lead to artifactual staining results.

. For plasmid-mediated transient transfection experiments, the cDNAs encoding syntaxin 2A or 2D or truncated versions of syntaxin 3 or 4 were inserted into the vector pcDNA4/TO and transfected into MDCK cells cultured on glass cover slips using the ExGen 500 transfection reagent (Fermentas, Inc., Hanover, Md.). After 24 hours, analysis of failed cytokinesis was carried out as described above.

Many SNAREs are closely related to each other, especially in the “SNARE” motifs (Weimbs, et al., 1997) which can lead to problems of antibody cross-reactivity. For example, polyclonal antibodies raised against GST-fusion proteins of any of the mammalian plasma membrane t-SNAREs syntaxin 2, 3 and 4 cross-react slightly with the other two syntaxins even after affinity-purification. A simple method to overcome this problem is competitive inhibition using lysates of bacteria that express the related syntaxin-GST fusion proteins (e.g. immuno-staining for syntaxin 2 would be carried out in the presence of syntaxin 3 and 4-lysates). Because the non-specific antibodies may be against both native and denatured antigen, a mixture of denatured and non-denatured lysates is added to the antibody solution before staining. The method comprises the following steps and compositions:

(1) Grow E. coli expressing GST-syntaxins under the appropriate conditions. Prepare a total cell lysate using a standard lysozyme protocol. Bacteria from a 1 liter culture will lead to approximately 25 ml of lysate; (2) add 250 μl SDS lysis buffer (0.5% SDS, 100 mM NaCl, 50 mM trithanolamine-Cl, 100 mM NaCl, 5 mM EDTA); (3) boil the above solution for 10 min.; (4) Add 250 μl Triton-dilution buffer (2.5% Triton X-100 500 mM trithanolamine-Cl, 100 mM NaCl, 5 mM EDTA); (5) Mix above solution, add 250 μl of non-denatured bacterial lysate and store at −80° C. in aliquots; and (6) add the above mixture at 4% to the primary anti-syntaxin antibody dilution during immunofluorescence staining.

A major strategy for defining the function of SNAREs is to specifically inhibit the function of an individual SNARE protein and measure the effects on the kinetics or fidelity of membrane trafficking pathways, the targeting of cargo proteins, or parameters of epithelial cell polarity. The difficulty lies in the fact that no ideal and simple method is available to inhibit SNAREs efficiently and specifically. Nature has provided clostridial neurotoxins—tetanus and botulinum toxins—which are highly specific metalloproteases that cleave and inactivate several SNARE proteins (Schiavo et al., 2000). However, most of these toxins cleave only neuronal SNAREs such as syntaxin 1, SNAP-25 and synaptobrevin which are not normally expressed in epithelial cells To make matters worse, clostridial neurotoxins can attach to and enter neurons but not non-neuronal cells. It is therefore necessary to introduce these toxins by other means. The same is the case for other inhibitory reagents such as antibodies and recombinant fragments of SNAREs. Two methods—using permeabilized cells or microinjection—can be used for introducing these non-membrane-permeable inhibitors into epithelial cells. An alternative strategy is to express dominant-negative inhibitors by gene transfer.

Dominant-negative inhibition by overexpression of SNAREs. It has been observed in several systems that the overexpression of a wild-type syntaxin causes inhibition of the trafficking pathway that the syntaxin is normally involved in. Examples are syntaxin 3 in MDCK cells (Low et al., 1998), syntaxin 5 in BHK-21 cells (Dascher and Balch, 1996), and syntaxin 4 in mast cells (Paumet et al., 2000). Although not wishing to be bound by theory, a plausible hypothesis of the observed inhibition is that the over-expression of one SNARE results in a stoichiometric imbalance with the other SNAREs involved in the same pathway. This may lead to the formation of non-productive, incomplete SNARE complexes and may cause one of the other functional SNAREs (or a regulatory factor) to become limiting. In any case, the effect appears to be quite specific as other trafficking pathways generally remain unaffected. However, successful inhibition requires relatively high levels of overexpression. For example, overexpression of syntaxin 3 (−10×over endogenous levels) by stable transfection in MDCK cells resulted in partial inhibition of biosynthetic trafficking to the apical membrane, as well as apical recycling, however, similar overexpression of syntaxin 4 had no measurable effect on any pathway (Low et al., 1998).

For this reason, an expression system should be chosen that results in high-level expression but can ideally also be regulated. Dasher and Balch used a vaccinia virus system which allows constitutive high-level expression (Dascher and Balch 1996). However, it is only useful for relatively short-term expression and may therefore be unsuitable to investigate long-term parameters such as development of epithelial cell polarity. The same is the case with the usual transient transfection approaches. A promising alternative is expression by stable transfection using a regulatable system such as those using the tetracycline repressor or transactivator. Also useful are adenoviral vectors that express the gene of interest under tetracycline control. The ability to regulate expression of the SNARE may be important because inhibition of any trafficking pathway may be potentially toxic.

In all cases it is important to verify that the over-expressed SNARE is still correctly targeted. Mistargeting of the SNARE of interest may compromise the specificity of the desired inhibition. Since too strong overexpression of any protein may result in its mis-localization, it is again desirable to be able to regulate the expression level. Accordingly, the methods of the present invention may include the step of verifying the over-expression of a particular SNARE (e.g. Syntoxin 2D) and the inhibition of other SNAREs (e.g. Syntoxin 2B)

Dominant-negative inhibition can also be achieved by expression of truncated SNAREs. There have been no systematic studies aiming at identifying domains of SNAREs that may be most potent and/or specific inhibitors. But is it generally believed that a non-membrane anchored truncation mutant will form complexes with its cognate SNAREs that are non-productive due to the lack of proper membrane attachment. A potential caveat is that non-membrane anchored SNAREs generally localize throughout the cytoplasm. Since SNARE-SNARE interactions—at least in vitro—are relatively promiscuous (Yang et al., 1999), the potential exists that a non-membrane anchored SNARE may be a less specific inhibitor than the same full-length SNARE when over-expressed.

Successful examples are the expression of the cytoplasmic domain of syntasin 4 in adipocytes (inhibits GLUT4 translocation (Olson et al., 1997) and the expression of the cytoplasmic domain of syntaxin 5 in BHK-21 cells (inhibits ER to Golgi transport (Dascher and Blach, 1996)). In both cases, vaccinia virus expression systems were used for relatively short-term experiments (−3-6 hours post infection). Expression of non-membrane anchored mutants of syntaxin 2 and endobrevin/VAMP-8 using a replication-deficient, tetracycline-regulatable adenovirus system very effectively inhibited the function of the respective SNAREs in MDCK cells. In this case, the cells could be monitored for periods up to 24 hours post infection. As an important control, the observed inhibitory effects could be eliminated by tetracycline-suppression.

These and still further objects as shall hereinafter appear are readily fulfilled by the present invention in an unexpected manner as will be readily discerned from the following detailed description of the preferred embodiments of the invention, especially when read in conjunction with the accompanying drawings.

The present invention is not to be limited in scope by the specific embodiments described which are intended as single illustrations of individual aspects of the invention, and any constructs, viruses, antibodies, toxins, or proteins which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims.

What is claimed:

1. A composition comprising: an effective amount of an inhibitor of a SNARE protein, said inhibitor effective in preventing cytokinesis in a population of cells.

2. The composition of claim 1 wherein said SNARE protein inhibitor is expressed in said cells.

3. The composition of claim 1 wherein said dominant-negative inhibitor is expressed using an virus vector with a regulatable promotor.

4. The composition of claim 1 wherein said dominant-negative inhibitor is expressed using an adenovirus vector with a tetracycline-regulatable promotor.

5. The composition of claim 1 wherein said SNARE protein inhibitor comprises a SNARE protein antibody

6. The composition of claim 1 wherein said inhibitor induces expression of a soluble isoforms of syntaxin 2.

7. The composition of claim 1, wherein said inhibitor induces expression of a soluble isoforms of endobrevin.

8. The composition of claim 6, wherein said soluble isoform of syntaxin 2 is selected from the group consisting of Syntaxin 2C and Syntaxin 2D.

9. The composition of claim 14, further comprising a pharmaceutically acceptable carrier.

10. A method of treating, preventing, or ameliorating a disease in a patient in need thereof comprising: administering to said pateint an effective amount of a composition effective in inhibiting SNARE function during cytokinesis.

11. The method of claim 10 wherein said composition comprises antibodies of SNARE proteins.

12. The method of claim 10 wherein said composition comprises antibodies of syntaxin 2.

13. The method of clam 10 wherein said composition comprises antibodies of endobrevin.

14. The method of claim 10 wherein said composition forms nonfunctional complexes with SNARE proteins.

15 The method of claim 10 wherein said composition is administered to said patient to induce over expression of syntaxin 2 isoforms in said patient.

16. The method of claim 10 wherein said composition is administered to said patient to induce over expression of endobrevin isoforms in said patient.

17. A method of forming binucleated cells comprising: administering to a collection of cells an effective amount of a composition to form binucleated cells from cells comprising said collection of cells, said composition comprising an inhibitor of a SNARE protein.

18. The method of claim 9 wherein said composition comprises a SNARE protein inhibitor.

19. The method of claim 10 wherein said composition comprises a SNARE protein inhibitor chosen from the group consisting of antibodies, DNA constructs, toxins, competing analogs of SNARE proteins, and combinations of any of these.

20. The method of claim 17, wherein said binucleated cells formed thereby are more susceptible to apoptosis.
